(19) United States
(12) Patent Application Publication
(10) Pub. No.: US 2009/0210062 A1
(43) Pub. Date: Aug. 20, 2009

(54) Orthopaedic Implants and Prostheses

(76) Inventors: John Thalgott, Las Vegas, NV (US); David T. Stinson, Woodinville, WA (US)

Correspondence Address: Beusse Wolter Sanks Mora & Maire, 390 N. ORANGE AVENUE, SUITE 2500, ORLANDO, FL, 32801, US

(21) Appl. No.: 12/034,139

(22) Filed: Feb. 20, 2008

(51) Int. Cl. A61F 2/44; A61B 17/04

(52) U.S. Cl. 623/17.16; 623/17.11; 606/301

(57) ABSTRACT

Disclosed herein are spinal implants particularly useful in interbody fusion surgery. One embodiment pertains to a plate configured to establish desired lordosis and/or disc height that may be implanted and secured to a superior and inferior vertebral body. The plate may be interlocked with a spacer component to form a single implant. Also disclosed is an anti-backout mechanism that helps prevent fixators from backing out upon securement of the plate in the spine. Kits comprising different sizes and inclination angles of components are disclosed, which can assist the surgeon in preoperatively assembling an implant to best fit in the surgical site of the patient.

### FIELD OF THE INVENTION

The present invention relates to orthopaedic implants and /or prostheses and instrumentation for their implantation. The invention is applicable to bone structures, particularly the cervical, thoracic and lumbar spine.

### GENERAL BACKGROUND

Spinal fusion for the management of lumbar degenerative disc disease has been available for several decades. The results of this procedure remain under constant scrutiny and progressive development. Anterior lumbar fusion was initially introduced in the early 1920s. Fibula and iliac struts, femoral rings and dowel, as well as synthetic metallic devices have been applied as fixation implements to aid in lumbar interbody fusion. Approaches to the spine have experienced similar evolutionary changes. Prior to the 1950s most anterior lumbar approaches were extensive transperitoneal exposures (i.e. through the membrane lining the walls of the abdominal and pelvic cavities). In 1957, Southwick and Robinson introduced the retroperitoneal approach (i.e., behind the peritoneum). Transperitoneal exposures (i.e., through the peritoneum) require incision of both the anterior and posterior peritoneum. In contrast, retroperitoneal exposures maintain the integrity of the peritoneum and approach the spinal column laterally behind the bowel and peritoneal contents. This has the advantage of less post-operative bowel problems. Additional changes in technique have seen the advent of minimally invasive approaches, including endoscopic and laparoscopic methods. Minimally invasive approaches are generally directed at one or two-level disease processes. Anterior lumbar interbody fusion (ALIF) may be useful in the treatment of unyielding low-back pain. The cause of this pain is often difficult to diagnose. Broad categories of pathology that may be associated with persistent low-back pain include degenerative disc disease, spondylolysis, spondylolisthesis or iatrogenic segmental instability.

Bones and related structural body parts, for example spine and/or vertebrae and/or intervertebral discs, may become crushed or damaged as a result of trauma/injury, or damaged by disease (e.g. by tumour, auto-immune disease), or damaged as a result of degeneration through an aging process. In many such cases the structure can be repaired by replacing the damaged parts (e.g. vertebra and/or discs) with a prosthesis or implant. A method of repair is to remove the damaged part(s) (e.g. vertebra and/or partial vertebra and/or disc and/or partial disc) and replace it with the implant or prosthesis such that the implant or prosthesis is free standing or fastened in position between adjacent undamaged parts (e.g. adjacent vertebrae).

Associated with this method of repair, is fusion of the bone structure where the implant or prosthesis is placed. Typically an implant or prosthesis may consist of a central space surrounded by a continuous wall that is open at each end (e.g. superior and inferior). This form of implant or prosthesis is thought to allow bone to develop within the central space, developing from each extremity of the implant or prosthesis towards the centre. Typically an implant or prosthesis shall be secured directly to a bone structure by mechanical or biological means.

While there has been an evolution of the shape of implants and some attempts to provide modular implants, the inventors have recognized that such changes have been relatively minor and have not fully contemplated cooperation between optimizing the surgical result and improving efficiency and safety of the operative procedure.

### SUMMARY

The subject invention is based on the inventors' recognition of several shortcoming of conventional implants, as well as an unfilled need for implants that are load bearing, and restore or maintain the lordotic angle and height of the intervertebral space. According to one embodiment, the invention pertains to a load bearing plate implant that is designed for insertion into the intervertebral space. In various embodiments, plate is geometrically configured for use with an anterior, anterolateral or lateral surgical approaches. For example, in some embodiments tailored for an anterior or anterolateral surgical approach, the plate includes a top surface and bottom surface such that a cross-section of a front to back longitudinal plane shows a tapering from an anterior side to a posterior side. This tapering assists in matching the anatomy of the intervertebral space in a sagittal plane thereby increasing the surface area of the footprint on both the superior and inferior vertebral bodies. Furthermore, when an anterior or anterolateral surgical approach is implemented, a cross section of the longitudinal plane of the plate may have a generally convex shape (a heightened body section with tapers down to lateral ends) which suitably matches the anatomy of the disc space. For a lateral surgical approach, a cross-section of the lateral to lateral longitudinal plane of the plate has a tapered or generally wedge-like geometric shape. The plate embodiment is especially versatile because it not only serves a load-bearing member in and of itself, but may be used in conjunction with a spacer (or cage) component. The spacer and plate may be rigidly engaged together or the plate may be used as a buttress without engagement to prevent shifting on a spacer positioned in the intervertebral space.

As indicated above, the plate embodiments may be especially adapted for different surgical approaches. FIG. 29 is provided which illustrates the basic direction of access to the intervertebral space for each of the primary surgical approaches. The anterior approach comprises an approach directly from the anterior vector of the vertebral body with 20 degree variability, the anterolateral approach is 45 degrees from the anterior vector with 25 degree variability and the lateral approach is 90 degrees from the anterior vector with 20 degree variability. Implant embodiments of the present invention facilitate easier, quicker and more precise surgical techniques that enable the restoration and re-establishment of spinal anatomy, lordosis and/or disc height. Implant embodiments of the present invention also are safer to use and increase the chances of a positive surgical outcome.

In certain embodiments, the plate is designed to address another problem associated with conventional spinal implants recognized by the inventors. This relates to the mode of securement of the implant to the vertebral body. For example, U.S. Pat. No. 7,232,464 ('464 patent, assigned to Synthes) teaches a spinal implant that comprises a body portion and a plate portion that is inset to the body portion. The '464 patent teaches that the boreholes of the plate should be threaded such that a bone screw may be rigidly screwed into the implant. The '464 patent is under the misapprehension that threading the screws into threads in the implant provides a preferred affixation. While not excluding the implementation of this type of affixation, the inventors take a contrary viewpoint concerning the mode of affixing the implant to the vertebral body and the association between bone, fixator (e.g., screw) and implant. Accordingly, in certain embodiments, the inner walls of the channels of the implant are not affixed to the fixator, such as by threads or otherwise. The fixator freely passes through the channel and is screwed into the vertebral body. As the fixator is tightened, this pulls the implant toward the vertebral body. Thus, the implant is secured to the vertebral body in a fashion analogous to the concept of interfragrnentary compression, which unifies the load path from the bone to the implant. It is the inventors' belief that this association between implant, fixator and bone is superior to that described in the '464 patent.

Another problem that the inventors have recognized with conventional implants is an absence of variability in the vector that the bone fixator (screw) may be directed for securement to the vertebral bodies relative to the angle of the implant. For example, the '464 patent described above discloses a number of boreholes through which the fixators are directed through and secured to the boreholes via threads. However, the vector of the bone screw is static. That is, the bone screw cannot move relative to the vector of the borehole. The inventors have recognized that this is a shortcoming in conventional design. Adjacent to the spinal column is critical vasculature for the body which runs down along the anterior portion of the spine. Further, the spinal nerves extend out laterally from the spine. Thus, a challenge for spinal surgeons is avoiding such vital anatomical structures during surgery as well as securing the implant so as to minimize possible interference between the implant or fixators and the vital anatomical structures subsequent to surgery. Accordingly, another implant embodiment comprises channels that allow for angular variability in the vector of the fixator is desired. FIG. 21 illustrates the angular variability or dynamism of the fixator allowed by the channel. This angular variability now provides surgeons with a level of adjustability with respect to where the fixators are secured and the orientation and placement of the implant relative to the fixators. This in turn will enable the surgeon to place the fixators in such a way as to minimize disrupting or damaging vasculature and nerves, whether intraoperatively or post-operatively, as well as adapt to a patient's unique anatomy. Increased safety and improved surgical outcomes are achieved.

In a specific embodiment, the channels of the implant are configured such that a fixator comprises angular variability of 40 degrees (see angle Z in FIG. 21) or less, preferably 25 degrees or less, around a central axis of the respective channel. The central axis pertains to a vector running through the centre of the channel.

In other embodiments of the invention, another problem associated generally with affixation in the spine is addressed: fixator back out. That is, after insertion into the vertebra, the fixator runs the risk of working loose and/or backing out of the vertebra. The consequence of backout or loosening of the implant or prosthesis includes loss of stability, potential risk to the patient and a separate costly operation. According to one embodiment, the subject invention pertains to a plate implant that comprises an anti-backout means to prevent backout of fixators. The concept of “backing out” is somewhat controversial, as some surgeons take the stance that it is a real phenomenon, while others think this is not a real risk. The inventors have realized that depending on the surgical site and the patient's anatomy, and surgeon preference, it may be beneficial to lock certain channels while keeping other channels unlocked. Thus, in certain implant embodiments, the anti-backout means pertains to a shiftable lock proximate to the channel opening. Each channel can be individually and independently closed following affixation of the fixator to bone. The fixators may be screws, pins, staples, darts, bollards or other suitable fixators. The ability of each channel to be individually locked provides options to surgeon depending on the placement of the implant and surgeon preference.

As already discussed above, a number of vital vasculatures and nerves are adjacent to and extend from the spine. The inventors have recognized that in circumstances where a portion of an implant protrudes from the intervertebral space this can cause a wearing down of vasculature over time. In extreme cases, this can result in a rupture of the vasculature and probable death. Accordingly, in certain embodiments, the implants are characterized as “no profile”, i.e., fully contained within the intervertebral space without protrusion. Prior art plates such as that discussed in U.S. Pat. No. 7,172,627 are typically designed for securement to the exterior wall of the vertebral body not the inner wall (see FIG. 27). One unique feature of plate embodiments of the present invention relates to their ability to be implanted into the intervertebral space (as opposed to overlaying the exterior wall of vertebral bodies and bi-directionally fixated into superior and inferior vertebral bodies. In certain advantageous embodiments of the invention, the implant is both no profile and allows bidirectional fixation (FIG. 28).

In a particularly advantageous embodiment, the present invention pertains to a plate having a top surface, bottom surface and side perimeter surface. The top and bottom surface include an engagement means for initially insetting the plate into the intervertebral space. The engagement means may take the form of one or more suitable raised protrusions, including but not limited to, keels, ridges, knobs, fins, serrations, and the like. The plate is typically tapped into the intervertebral space wherein the engagement means is inset into the vertebral body.

According to another embodiment, the invention pertains to an interbody implant that includes a spacer and a plate. The spacer has a top surface, bottom surface and side perimeter surface. The side perimeter surface has at least one fixator portal defined therein. The plate has a side perimeter surface, top surface and bottom surface and has at least one channel defined therethrough. A portion of the plate side perimeter surface may be configured to rest adjacently against at least a portion of the spacer side perimeter surface such that the at least one channel overlays the at least one fixator portal. The spacer and plate may be secured together to form a unitary implant.

According to another embodiment, there is provided a kit of parts for use in assembling a spinal implant or prosthesis, comprising: a plurality of implant members for insertion into an intervertebral space, the implant members being of a range of sizes and/or shapes to suit different sizes/shapes of intervertebral space. The implant members are configured to interconnect to form a suitable implant which takes into account the dimensions of the particular subject treated. One exemplary means for the engageable interconnection of implant members comprises a mechanical joint such as a push or snap-fit connection.

Optionally, another embodiment of the invention pertains to a method for surgically implanting an implant in an intervertebral space between a superior and inferior vertebra. The method includes the positioning of a plate into the intervertebral space. The plate has side perimeter surface, a top surface and a bottom surface. The plate also has a first and second channel. A first fastener is passed through said first channel and secured into the superior vertebra; and a second fastener is directed through said second channel and secured into said inferior vertebra.

In certain embodiments, bone ingrowth materials are implemented which may be disposed within various cavities defined in the embodiments, and/or used as coating the components. Bone ingrowth materials may comprise known bioactive materials including but not limited to BMP or other suitable growth factors, allograft bone with/without stem cell enrichment, calcium phosphate, and/or autograft bone. See U.S. Pat. Nos. 6,899,107 and 6,758,849 for general information on osteoinductive, osteoconductive and/or osteogenic materials and implants. Further, in alternate embodiments, bone ingrowth materials are made of solid materials which are pre-cut and pre-shaped and are conjoined with other implant components during assembly of the implant.

It is an advantage that the practitioner can select an appropriate size of spacer and appropriate sizes of plates from the kit of parts to suit the particular size and shape of the space into which the implant or prosthesis is to be inserted. In addition, the practitioner can adjust the size of the spacer and/or plate selected. Not only do sizes vary from patient to patient, but also the size and shape of the space varies according to the location in the spine.

These and other features and embodiments are described in further detail below.

### BRIEF DESCRIPTION OF THE DRAWINGS

FIG. 1 shows a perspective view of a spinal implant embodiment useful for an anterior surgical approach having a spacer component and plate component.

FIG. 2 shows a side perspective view of a spacer embodiment.

FIG. 3 shows a top perspective view of a disassembled spinal implant embodiment.

FIG. 4 shows an anterior side perspective view of an assembled spinal implant embodiment.

FIG. 5 shows a posterior side perspective view of an assembled spinal implant embodiment.

FIG. 6 shows a top view (a) anterior view (b) side perspective view (c) and side view (d) of a spinal implant embodiment.

FIGS. 7 and 8 show a spinal implant embodiment secured to a superior and inferior vertebral bodies.

FIG. 9 shows a side perspective view of a buttress plate embodiment.

FIG. 10 shows a side perspective view of a one-hole buttress plate embodiment.

FIG. 11 shows the buttress plate embodiment of FIG. 9 attached to a vertebral body superior to an implant.

FIG. 12 shows the buttress plate embodiment of FIG. 10 attached to a vertebral body superior to an implant.

FIG. 13 shows a side perspective view of a spinal implant embodiment having a spacer component and plate component useful for a lateral surgical approach.

FIG. 14 shows a side perspective spacer component embodiment.

FIG. 15 shows a side perspective view of a disassembled spinal implant embodiment.

FIG. 16 shows a side perspective view of a spinal implant embodiment.

FIG. 17 shows a posterior perspective view of a spinal implant embodiment.

FIG. 18 shows a side view of a spinal implant secured between a superior and inferior vertebral body.

FIG. 19 shows a see-through side view of an embodiment secured between a superior and inferior vertebral body.

FIG. 20 shows a top view (a) a front view (b), a perspective view (c) and a side view (d) of a spinal embodiment.

FIG. 21 and 22 are cross-sectional views of the screw arrangement as assembled in the buttress plates of FIG. 9 or 10.

FIG. 23 is a cross-sectional view of the screw arrangement as assembled in the embodiment of FIG. 1.

FIG. 24 shows a modified buttress plate secured to a vertebral body.

FIG. 25 shows a cross-section of a buttress plate arrangement.

FIG. 26 shows a cross-section of the arrangement of FIG. 1.

FIG. 27 shows a side view of a prior art implant that has a profile that extends outside of the intervertebral space.

FIG. 28 shows a side view of an implant of the present invention that has no profile.

FIG. 29 shows a top view of a vertebral that illustrates surgical approaches that are enabled by embodiments of the invention.

### DETAILED DESCRIPTION

### EXAMPLE 1

With reference to FIGS. 1-8 a spinal implant embodiment will now be described. FIG. 1 shows an anterior perspective view of a spinal implant 10 embodiment that includes a plate component 12 and a spacer component 14. The spacer component 14 comprises a cavity 20 defined therein for disposing a bone ingrowth material. The plate component 12 comprises a keel 40 having an apex that serves to penetrate the bone surface of a vertebral body. It should be noted that the plate 12 may be utilized with or without the spacer component 14.

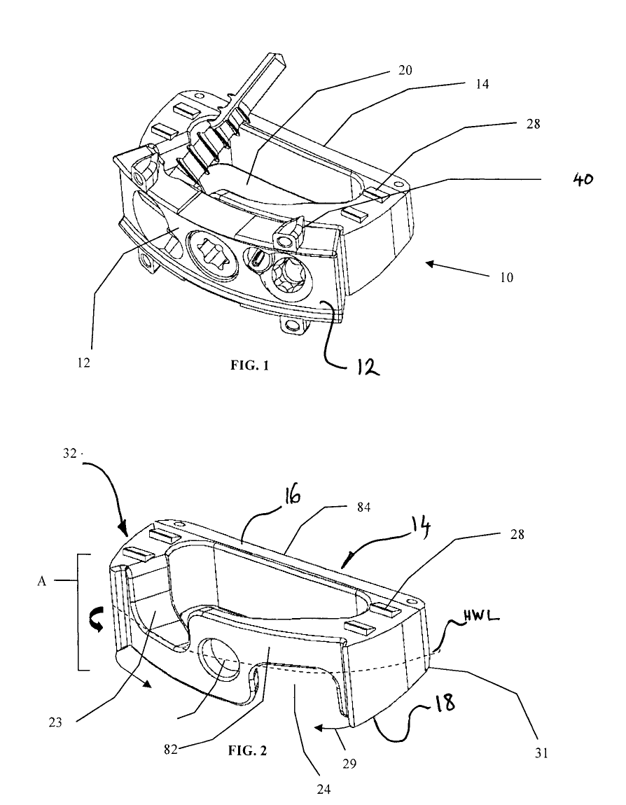

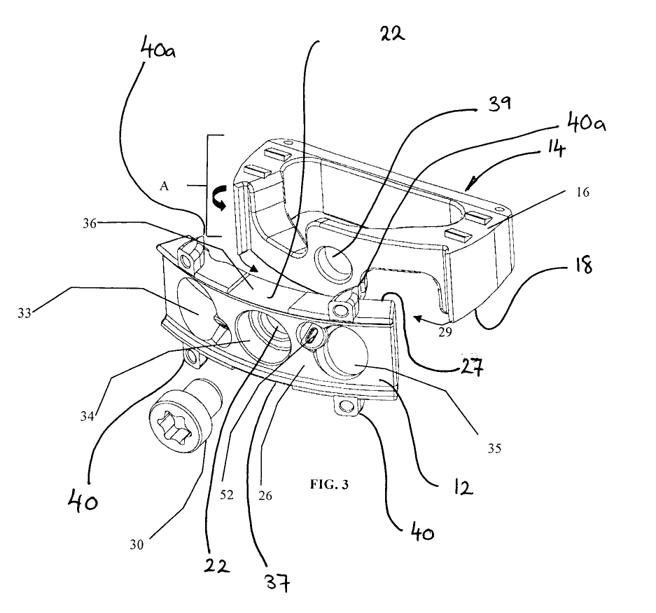

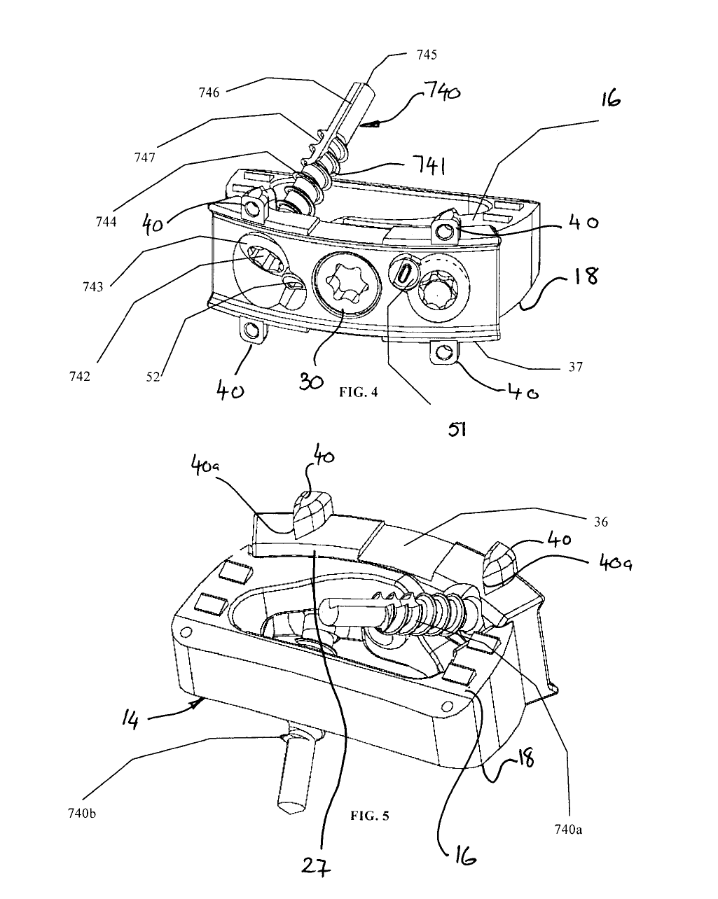

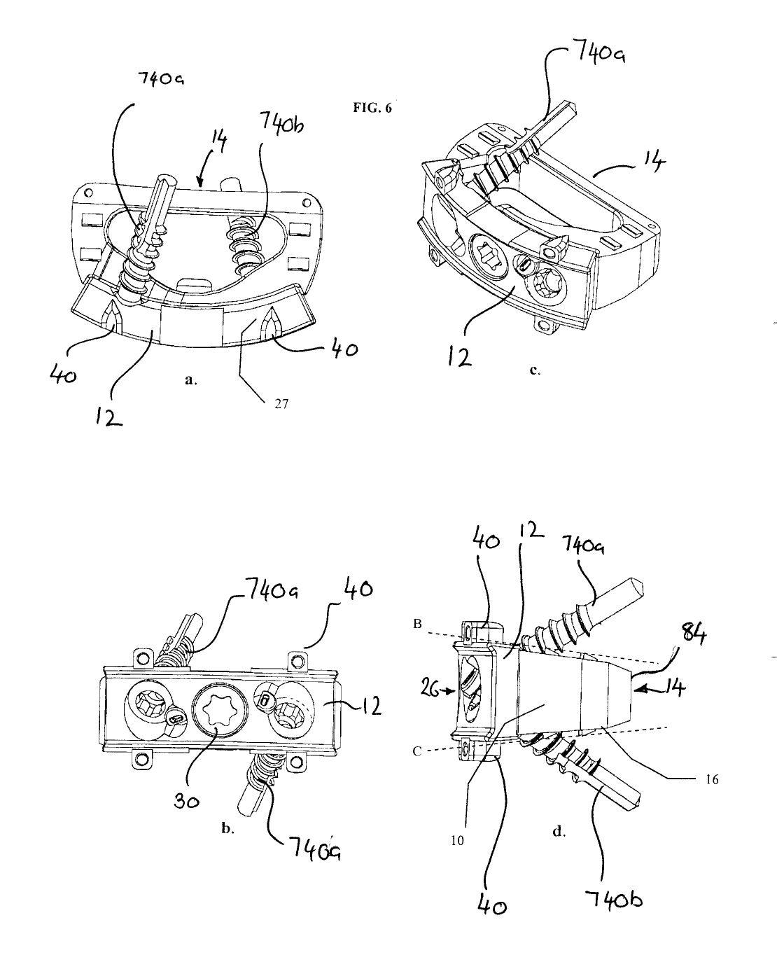

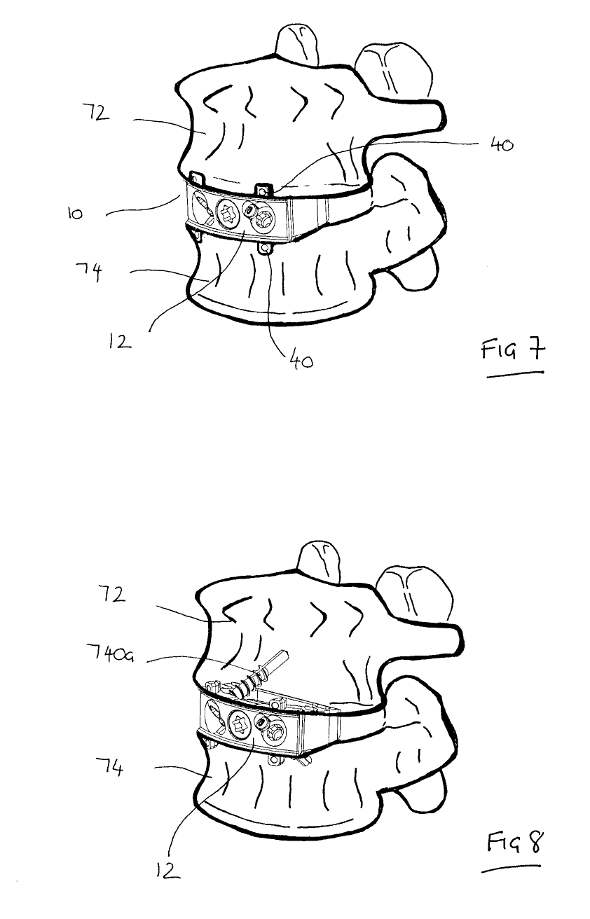

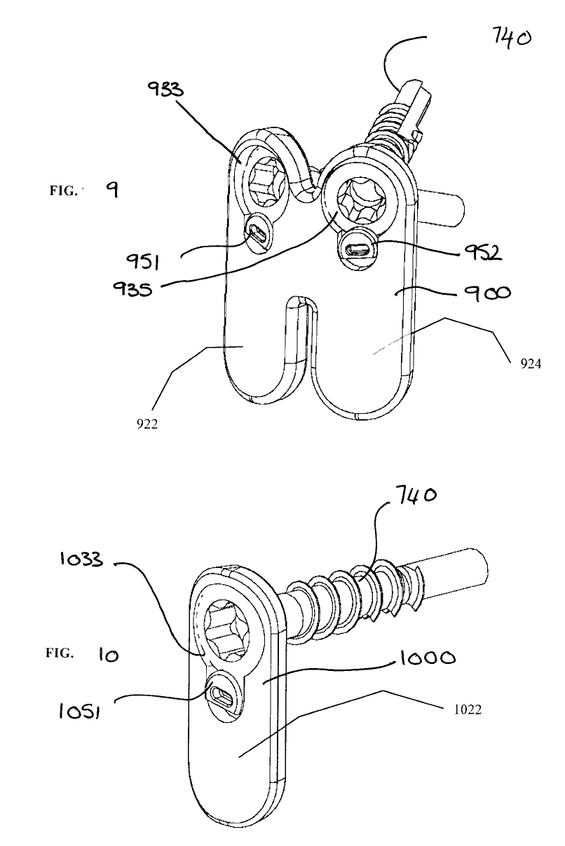

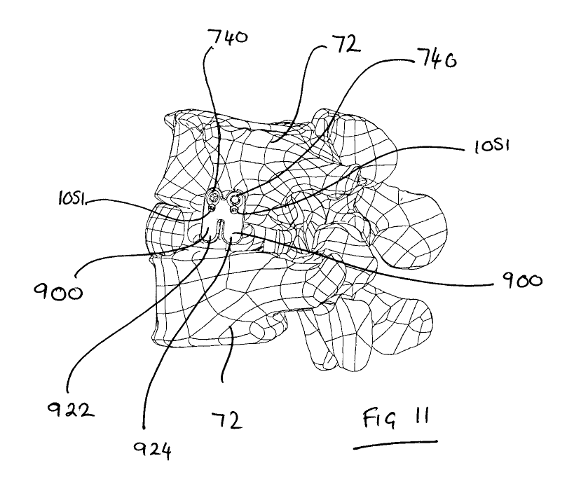

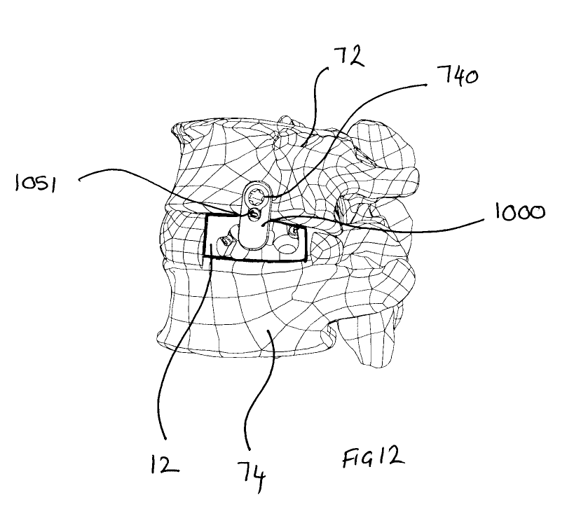

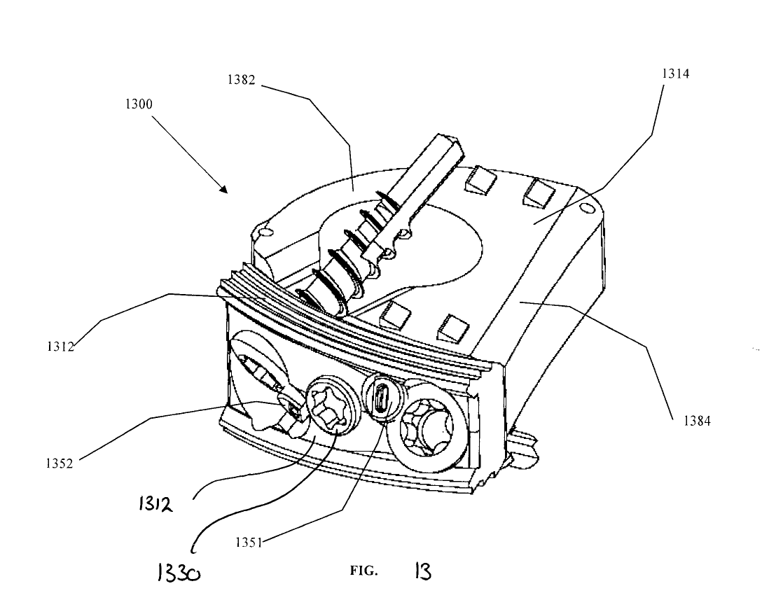

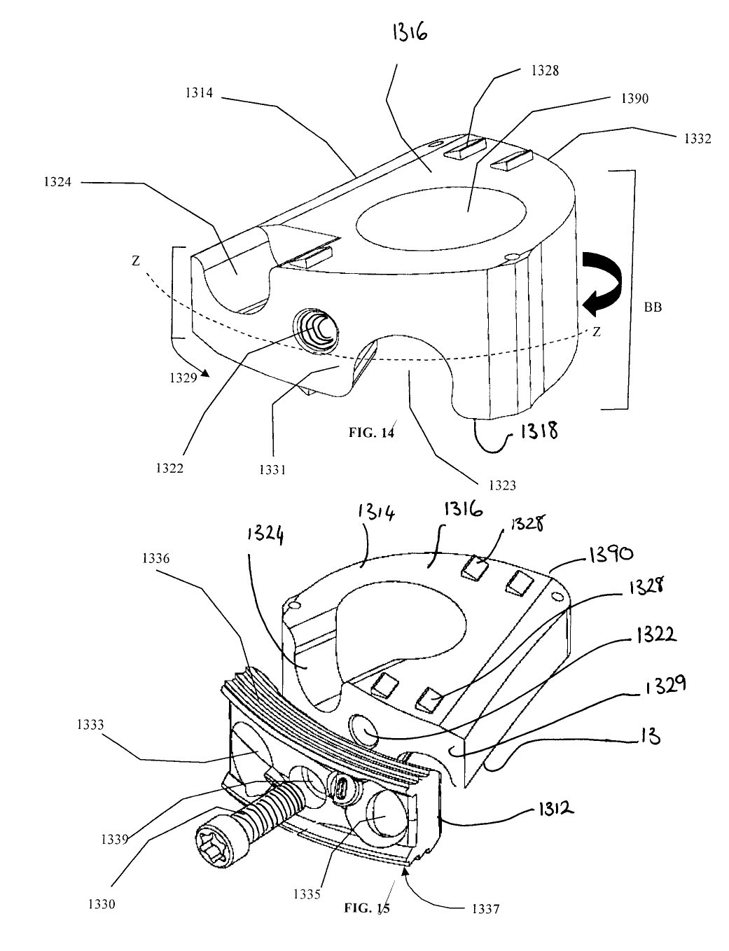

FIG. 2 shows a perspective view of the spacer component 14. The spacer component 14 has an anterior body portion 82 and a posterior body portion 84. The spacer component 14 also has a lateral end 31 and a lateral end 32. The spacer component has a top surface 16 and a bottom surface 18 (see FIGS. 4, 5 & 6d) and a side perimeter surface A. The anterior body portion 82 has an anterior side 29. On the anterior side 29 of the anterior body portion 82 is defined an interlocking aperture 22, a portal 23 and a portal 24. Also shown on the top surface 16 and bottom surface 18 of the spacer component 14 are projections 28 which assist in gripping a superior and inferior vertebral body. FIG. 2 also shows a half way line HWL and it will be appreciated that the portal 23 has an opening that is positioned on the top half of the anterior side 29 of the anterior body portion 82 which is open to the upper surface 16. Conversely, portal 24 has an opening that is positioned on the bottom half of the anterior side 29 of the anterior body portion 82 and is open to the lower surface 18.

FIG. 3 shows a disassembled perspective view of the plate component 12 and spacer component 14. The plate component has a top surface 36, a bottom surface 37 (see FIG. 5) an anterior side 26 and a posterior side 27. Defined in the plate component is a channel 33 and a channel 35. Further, an interlocking aperture 39 is defined in the plate component 12. The plate component 12 is brought together with the anterior side 29 of the spacer component 14 such that the channel 33 aligns with the portal 23, the channel 35 aligns with the portal 24 and the interlocking aperture 39 aligns with interlocking aperture 22. A threaded interlocking member 30 is positioned through the interlocking apertures 22 and engages with a correspondingly threaded aperture 39 on the spacer 14 such that the plate component 12 is secured to the spacer component 14. It will be appreciated that other forms of interlock may be used such as, for example, bayonet fittings or twist locks. Also shown are projections 40 to assist in gripping a superior and / or inferior vertebral body. Such projections may comprise a ridge, keel, fin, knob, or a combination thereof. In a preferred arrangement, the projections comprise keels having sharp leading edges 40a which, in operation, are driven into the vertebral body during assembly of the implant in a patient.

Turning now to FIG. 4 which includes a self-taping, self-drilling screw 740 positioned in channel/portal 33/23. The screw 740 comprises an elongate body 741 comprising a proximal end 743 and distal end 745. The distal end 745 comprises a drill region 746 which is configured to initiate drilling a whole into bone. The elongate body comprises a taping region 747 which is configured to initiate taping into bone and a threaded region 744 which is configured to screw into bone. A driver 742 is defined in the proximal end 743 of the screws 740. The driver may take many suitable forms. Driver 742 is configured as a hex drive. FIG. 4 also shows shiftable locking components 51, 52, in a closed state which serve to prevent backing out of screws 740, as will be described in more detail later herein. The shiftable locking components 51, 52 are fixed to the plate component 12 proximate to the channels 33, 35 such that they may be pivoted or otherwise shifted to cover the opening of the channels 33, 35, i.e., a closed state.

FIGS. 5 and 6 illustrate the assembled arrangement and from which it will be appreciated that screw 740 passes through the upper portal 23 before it penetrates an upper plane B (FIG. 6d) of the implant itself. The lower screw 740 passes through the lower plane C in like manner. The portals 23, 24 and channels 33, 35 are sized and configured such that the screw 740 passes through at the anterior side 26 and is directed on an upward angle to traverse a plane of the top surface of the implant B, or a downward angle to transverse a plane of the bottom surface of the implant C. Further, the size and configuration of the portals and channels are such that there is 40 degrees or less, typically 25 degrees or less of angular variability around a central axis of the passage formed by the channel/portal combination. The portals 23, 24 shown represent a gap that opens to the top surface 16 or bottom surface 18 of the spacer component 14 and ends at about the half-way line. It is contemplated that portals could be an enclosed aperture as well, so long as the proper upward and downward angles are achieved for the screws 740. Typically, the opening will be predominantly positioned either on the top half or bottom half of the spacer component 14. FIG. 6 shows a top view 6a, a front view 6b, a side perspective view 6c and a side view 6d of implant with screws positioned therethrough. FIG. 6d shows how the implant 10 tapers down from the anterior side 26 of the plate 12 to the posterior side of the posterior body portion 84, see dashed lines B, C.

FIG. 7 shows the implant 10 secured to a superior 72 and inferior 74 vertebral body. The driver head 742 of screw 740a is shown which has been turned to cause the screw 740 to penetrate the verterbral body 72. FIG. 8 shows a see through perspective view of the implant 10. The implant 10 is secured to the superior vertebral body 72 by screw 740a and secured to the inferior vertebral body 74 by screw 740b. As described above, the plate component 12 rests adjacent to the side perimeter surface A (FIG. 3) of the spacer component 14. In many embodiments, the spacer component has a contoured portion and the plate component is configured to mirror the contoured portion on the side which rests against the spacer component. For example, the spacer component has a side perimeter surface that includes a shape including, but not limited to, a bend, rounded corned, apexed corner, or curve, or straight portion between a bend apexed corner or curve. The term “generally” in relation to curves or straight portions is understood to mean that portion in question is 80 percent or more, or 90 percent or more, curved or straight depending on the situation. In the example illustrated in FIGS. 1 to 8, the contoured portion is generally arcuate, or curved, along the anterior side 29 of the anterior body portion 82. The posterior side 27 of the plate component 12 is configured to mirror this bend. This allows for solid and securing contact between the plate and spacer components 12, 14.

### EXAMPLE 2

FIG. 9 shows a two-hole buttress plate 900 and FIG. 10 shows a one-hole buttress plate 1000 suitable for securing an implant within a vertebral cavity and particularly suitable for adding a further degree of security of fixing to the implant embodiments shown herein. The buttress plates 900 and 1000 are designed to attach to a vertebral body that is either superior to an intervertebral space into which an implant has been positioned such as that shown in FIGS. 11 and 12, or inferior to the intervertebral space into which an implant is positioned, or could be more than one with one buttress plate superiorly secured and one inferiorly secured. The buttress plates 900, 1000 may be secured with any suitable fastener, but are advantageously secured with the screws 740 such as that described above in relation to FIGS. 4 and 5, that are passed through either two holes for plate 900 or one hole for plate 1000 (holes hidden underneath head of screw). Figures. 9 and 10 also shows shiftable locking components 951, 952, or 1051 which are associated with the plate 900 or 100 such that they may be individually and separately shifted to obstruct the proximate channel 933, 935, or 1033 respectively. The shiftable locking components serve to prevent backing out of screws 740 and are described in more detail later herein. The two-hole implant 900 comprises two body portions 922 and 924 which extend across the periphery of the intervertebral space, whereas the one hole plate 1000 comprises one body portion 1022. The buttress plate serves to further facilitate safe, securement of the implant. Depending on the implant used, shifting of the implant can occur such that it is urged to protrude out of the intervertebral space. In circumstances where this protrusion is not desired, the buttress plates 900 and 1000 add an additional precautionary measure. FIGS. 11 and 12 illustrate the single and double buttress plates attached to a superior vertebral body and from which it will be appreciated that the screws 740 are secured to the vertebral body whilst the protruding body portions 922 and 924 cover the implant back plate 12 and provide a further degree of security to the implant location within the vertebral body. It will also be appreciated that the buttress plates may be used on their own and or with other implants not described herein.

It will be appreciated that Buttress plate embodiments may be made of a material possessing a degree of flexibility such that they may be bent to conform to the contours of a patient's given anatomy. Also, it is noted that the buttress 900, 1000 may comprise shiftable locking components 951, 952 similar to that described for the above embodiments.

### EXAMPLE 3

Turning now to FIG. 13, a side perspective view of an implant 1300 is shown, which is particular useful for a lateral surgical approach. The implant 1300 comprises a spacer component 1314 and a plate component 1312. The spacer component 1314 comprises an anterior body portion 1382 and a posterior body component 1384. The plate component 1312 comprises cam locks 1351, 1352 which assist in preventing “backing out” of screws passing through channels in the plate component 1312, which is discussed in further detail below.

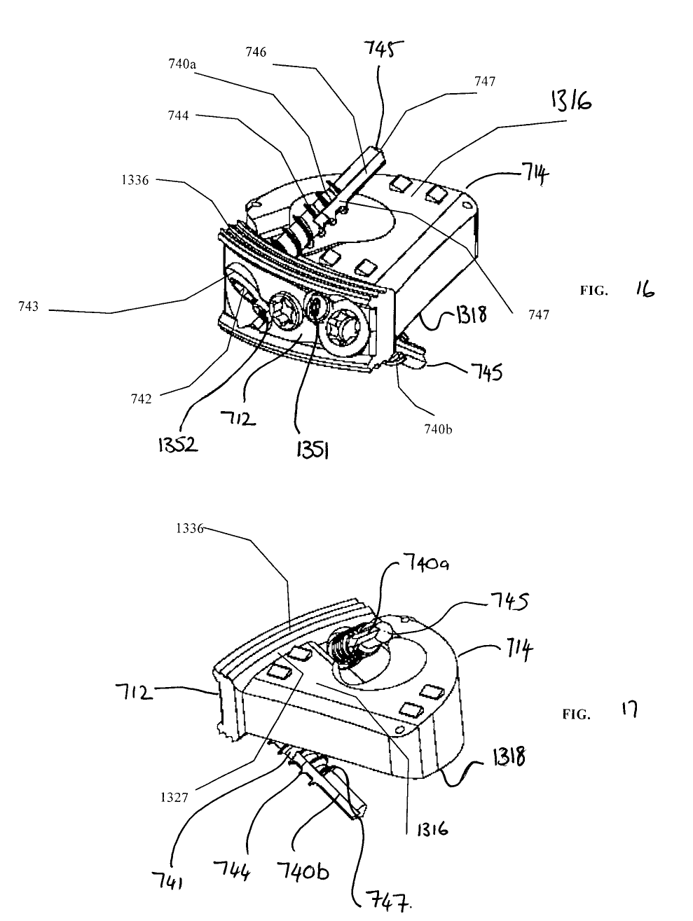

FIG. 14 shows a side perspective view of the spacer component 1314. The spacer component 1314 has a first lateral end 1331 and a second lateral end 1332. The spacer has a side perimeter surface as depicted by the arrows. Defined in the first lateral end 1331 are a first fixator portal 1323 and a second fixator portal 1324 which open at the side surface 1329 of the first lateral end 1331. Again, the portals are open to the immediately adjacent planar upper or lower surfaces 1316 and 1318 which is best seen in FIGS. 16 and 17. The spacer component 1314 comprises a cavity 1390 defined therein for disposing a bone ingrowth material and includes a side perimeter surface BB. Also on the side of the first lateral end 1331 is defined a threaded interlocking aperture 1322 for receiving a suitably threaded locking screw 1330, as discussed above with reference to FIG. 3. Also shown on the top surface 1316 and bottom surface 1318 of the spacer component 1314 are projections 1328 which assist in gripping a superior and inferior vertebral body. Also shown is a half-way line ZZ and from which it will be appreciated that the portal 1324 has an opening that is positioned on the top half of the lateral side 1329 of first lateral end 1331. Conversely, portal 1323 has an opening that is positioned on the bottom half of the lateral side 1329 of the first lateral end 1331.

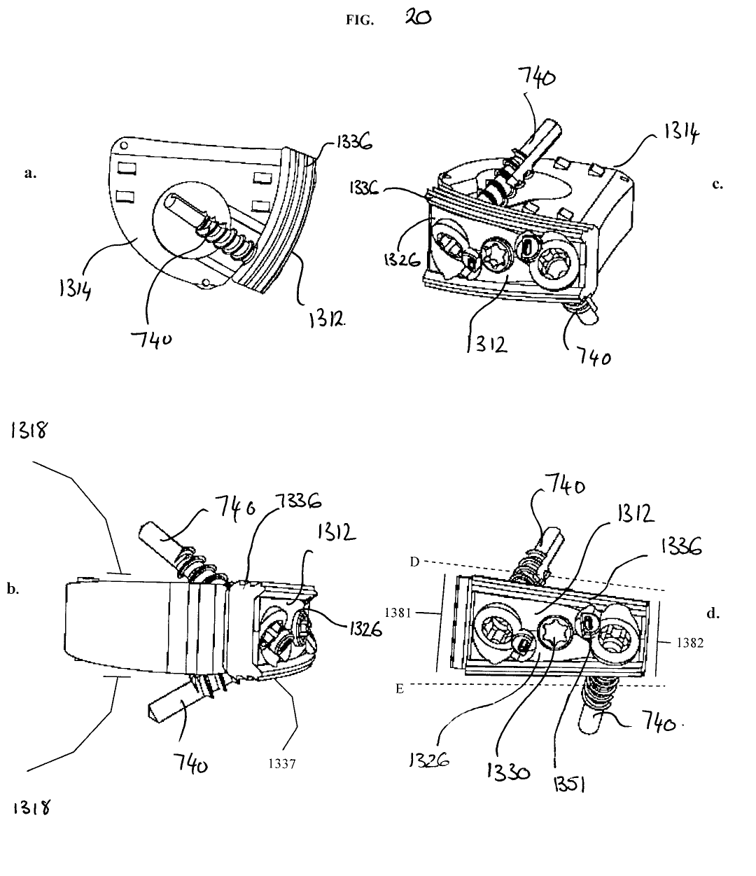

FIG. 15 shows a disassembled perspective view of the plate component 1312 and spacer component 1314. As with other plate embodiments described herein, the plate component 1312 may be used with or without the spacer component 1314. The plate component 1312 has a top surface 1336, a bottom surface 1337 (see FIG. 20b) lateral side 1326 and a medial side 1327 (see FIG. 17). Defined in the plate component is a channel 1333 and a channel 1335. Further, an interlocking aperture 1339 is defined in the plate component 1312. The plate component 1312 is brought together with the lateral side 1329 of the spacer component 1314 such that the channel 1333 aligns with the portal 1324, the channel 1335 aligns with the portal 1323 and the interlocking aperture 1339 aligns with interlocking aperture 1322. A threaded interlocking member 1330 is positioned through the interlocking apertures 1322, 1339 and engaged such that the plate component 1312 is secured to the spacer component 1314. Other forms of interlocking may be provided and include but are not limited to bayonet fittings and click fittings. Ridges 1336 are provided on the upper and lower surfaces of plate 1312 and may be angled rearwardly relative to the direction of assembly. Such ridges may act as interference ridges which engage with any adjacent vertebral structure such as to assist with the securing of said assemble relative thereto. The rearward angling will make the insertion easy but removal more difficult.

FIG. 16 shows a self-taping, self-drilling screw 740a positioned in channel/portal 1333/1324 whilst screw 740b is shown in channel/portal 1335/1323. Each screw 740 comprises an elongate body 741 comprising a proximal end 743 and distal end 745. The distal end 745 comprises a drill region 746 which is configured to initiate drilling a whole into bone. The elongate body comprises a taping region 747 which is configured to initiate taping into bone and a threaded region 744 which is configured to screw into bone. A driver 742 is defined in the proximal end 743 of the screws 740. The driver may take many suitable forms. Driver 742 is configured as a hex drive. FIG. 16 also shows shiftable locking components 1351, 1352, which serve to prevent backing out of screws 740. The shiftable locking components 1351, 1352 are shown in a closed state and are fixed to the plate component 1312 proximate to the channels 1333, 1335 such that they may be pivoted or otherwise shifted to cover the opening of the channels 1333, 1335, i.e., a closed state.

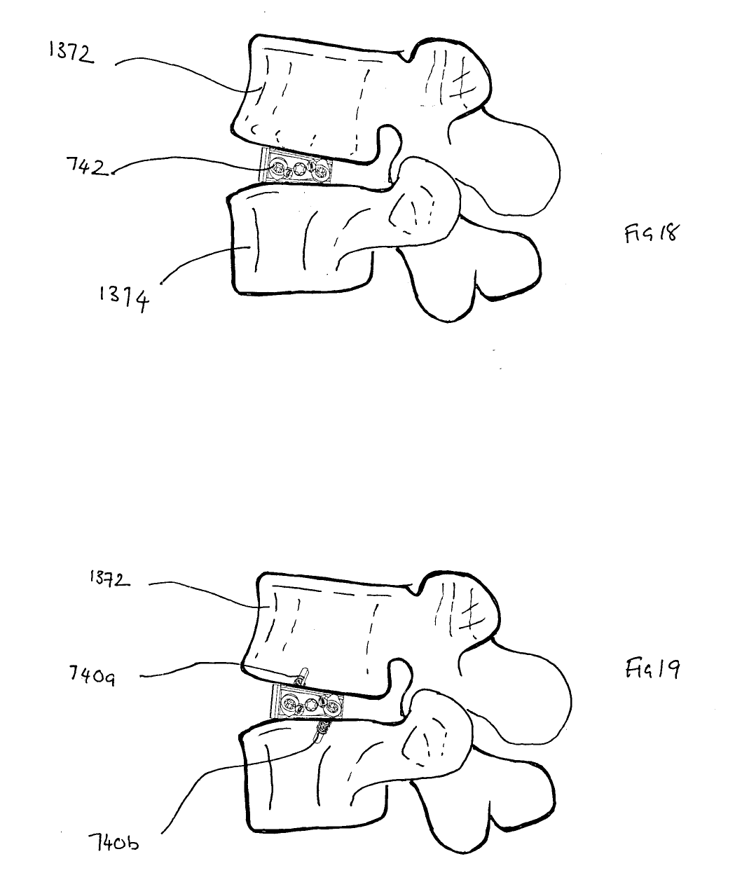

FIG. 18 shows the implant 1310 secured to a superior 1372 and inferior 1374 vertebral body. The driver head 742 of screw 740a is shown which has been turned to cause the screw 740 to penetrate the verterbral body 1372. FIG. 19 shows a see through perspective view of the implant 1310. The implant 1310 is secured to the superior vertebral body 1372 by screw 740a and secured to the inferior vertebral body 1374 by screw 740b.

FIG. 20 shows a top view 20a, a front view 20b, a side perspective view 20c and a side view 20d of implant with screws positioned therethrough. FIG. 20d shows how the implant 13 10 tapers down from the anterior side 1381 of the implant 13 10 to the posterior side 1382 of the implant, see dashed lines D, E. The portals 1323, 1324 and channels 1333, 1335 are sized and configured such that the screw 740 passes through at the lateral side 1326 and is directed on an upward angle to traverse a plane of the top surface of the implant D, or a downward angle to transverse a plane of the bottom surface of the implant E. Further, the size and configuration of the portals and channels are such that there is 40 degrees or less, typically 25 degrees or less of angular variability around a central axis of the passage formed by the channel/portal combination. Details of the screw and portal arrangement are shown in full later herein. The portals 1323, 1324 shown in FIG. 14 represent a gap that is open to the top surface 1316 or bottom surface 1318 of the spacer component 1314 and ends about at the half-way line. It is contemplated that portals could be an enclosed aperture as well, so long as the proper upward and downward angles are achieved for the screws 740. Typically, the opening will be predominantly positioned either on the top half or bottom half of the spacer component 1314. This arrangement effectively provides good freedom of movement for the bone fixators whilst also providing the wall portion surrounding said opening with the required strength and mass to support the plate and carry the vertebral loading.

As described above, the plate component 1312 rests adjacent to the side perimeter surface BB of the spacer component 1314 and is curved to match the profile thereof. In many embodiments, the spacer component has a contoured portion and the plate component is configured to mirror the contoured portion on the side which rests against the spacer component. For example, the spacer component has a side perimeter surface that includes a shape including, but not limited to, a bend, rounded corned, apexed corner, or curve, or straight portion between a bend apexed corner or curve. The term “generally” in relation to curves or straight portions is understood to mean that portion in question is 80 percent or more, or 90 percent or more, curved or straight depending on the situation. In the example illustrated in FIGS. 13-20, the contoured portion is curved all along the lateral side 1329 of the. The posterior side 1327 of the plate component 1312 is configured to mirror this bend. This allows for solid and securing contact between the plate and spacer components 1312, 1314.

### EXAMPLE 4

### Screw Mounting

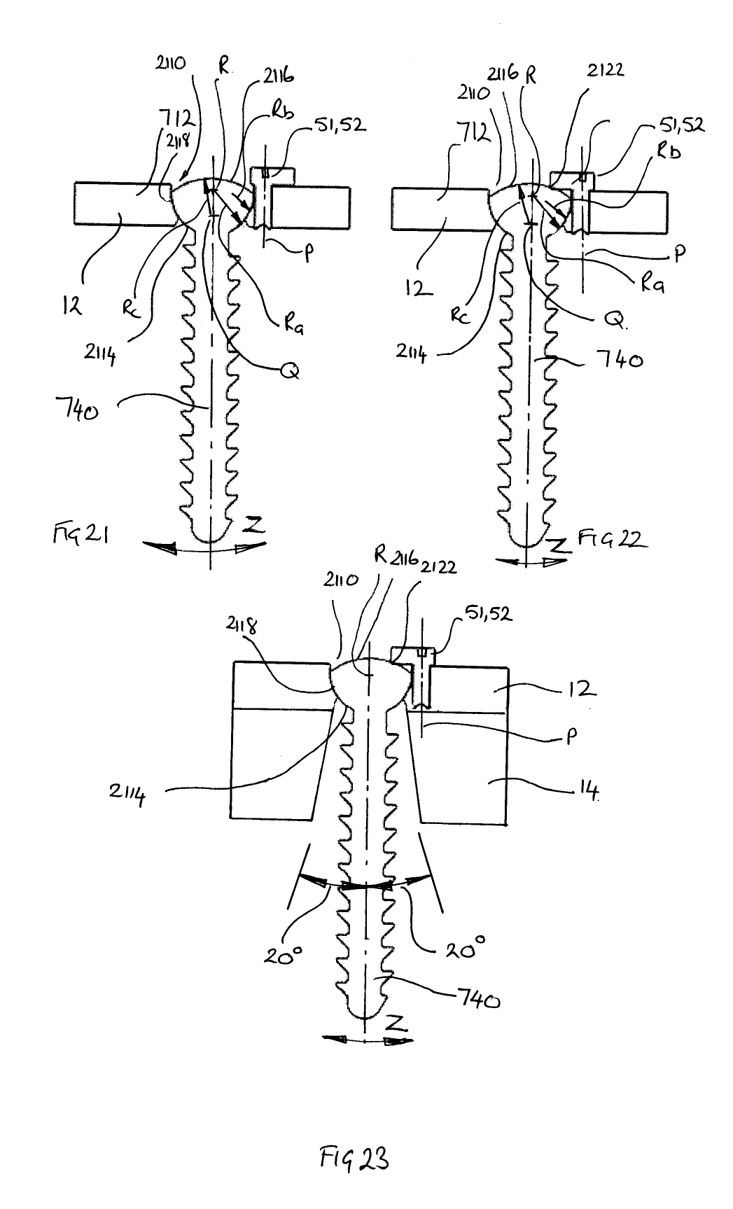

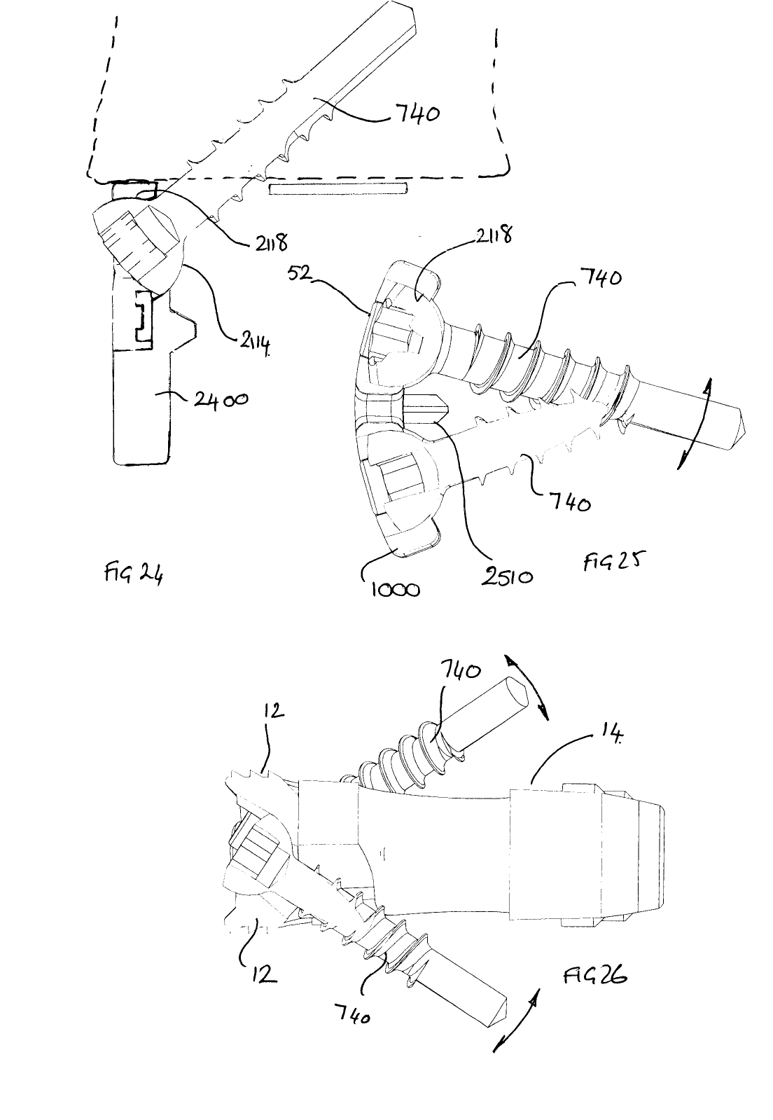

FIGS. 21 to 23 illustrates the bone fixation device 740 and locking components 51, 52 in more detail and from which it will be appreciated that the locking component 51, 52 is rotatable about axis P between a first position shown in FIG. 21 where it acts to unobturate the channel 2110 and a second position shown in FIG. 22 where it acts to engage with the head 2112 and prevent the screw 740 from backing out of the channel 2110. The locking component shown comprises a generally circular component having a flattened side which acts to form an opening when rotated to a suitable position. For further details please see the earlier figures. The screw head 2112 further includes a curved bottom surface 2114 having a radius Ra extending from point R and a curved top surface portion and having a radius Rc extending from point Q. The aperture itself is provided with an upper portion 2118 having a radius of curvature Rb matching or approximating that of Ra and an optional bottom portion 2120 (FIG. 23) which diverges, thereby to ensure adequate clearance for any angular movement of the screw 740. Radius Ra is selected such as to allow the screw 740 to pivot in the aperture whilst maintaining contact with the upper curved surface 2118. The upper curved surface 2112 is provided with a radius of curvature which may match that of the lower surface such that whenever the screw is pivoted the locking component 51, 52 will always be able to rotate into contact with the surface 2112 such as to cause said component to initiate a point contact at point 2122 and lock said locking component thereto such as to prevent movement of said screw out of said aperture. This is in contrast with the known art which merely acts to obdurate the aperture without actually engaging with the screw itself. It will be appreciated that radius Rc may be selected to be the same as radius Ra and that both may share a common origin such as to ensure a consistent and even clamping effect when the locking component 51, 52 is engaged with the head portion 2116.

FIG. 24 is a side view of a no-profile buttress plate 2400 of the same general construction as those described above with reference to FIGS. 9 and 10 save for the fact that it is provided with a top portion 2410 which extends under (or over) a vertebral body 2412 to which it is to be fitted. The screw 740 is angled upwardly (or downwardly) into the vertebra but otherwise is located in and secured to the buttress plate in the manner described previously. It will, however, be noted that the locking mechanism 51, 52 is recessed into the buttress plate such as to ensure adequate location with respect to the head of screw 740 and that the plate may well need to be deeper such as to accommodate the angular positioning of the screw 740.

FIG. 25 provides a plan view of a double buttress plate of FIG. 9 and illustrates how the screws 740 may be angled relative to each other so as to maximise the security of attachment. Also shown is a keel 2510 which will act to penetrate into a vertebra when the plate is attached thereto, thereby to improve the security thereof still further and prevent subsequent rotation of the plate after installation. This feature may also be included in the single buttress plate arrangement of FIG. 10.

FIG. 26 provides a cross-sectional view of the implant arrangement of FIG. 4 and illustrates the extent to which screw 740 is free to be angled relative to plate 12. This angular variation is important as it gives the surgeon much more freedom of choice over the angle that the screw and can facilitate good plate securing whilst also avoiding the screw being directed into or near undesirable areas.

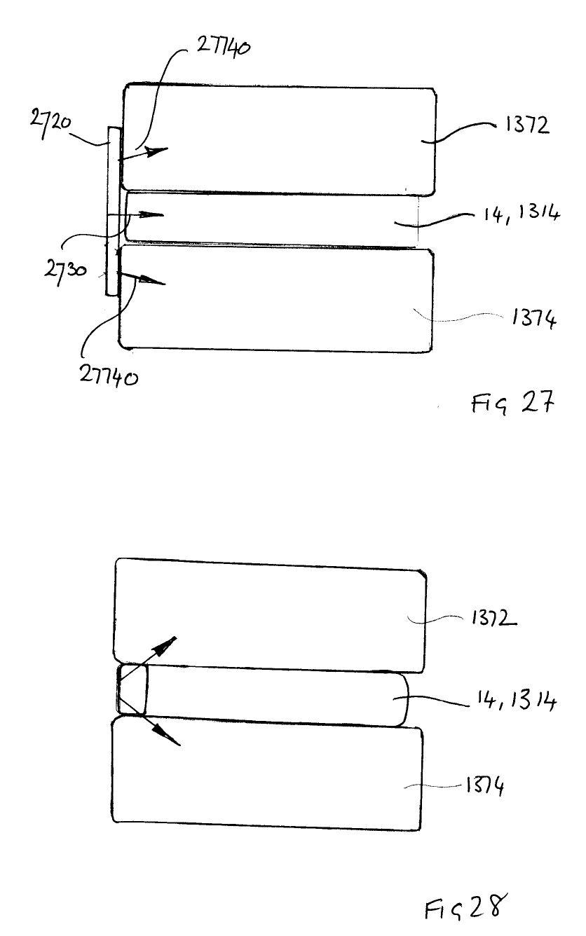

FIGS. 27 illustrates a cage arrangement well known in the prior art in which an implant shown generally at 14, 1314 is secured in position by a relatively low profile plate 2720 provided on the outside of the vertebra and bridging two adjacent vertebra such as to prevent the implant from migrating out of the inter-vertebral gap. The plate may be secured by screws shown at 27740 and may also be secured to the implant by means of a screw or other such device shown schematically at 2730. Whilst such an arrangement does not provide a “no-Profile” method of securing an implant it can be adequate in some circumstances and may lend itself to use with the present arrangements where the screw 2730 is secured to the implants of the present invention, thereby avoiding or supplementing the use of screws 740 of the above arrangements. It will, therefore, be appreciated that screws 740 may be eliminated in some circumstances and are important but not absolutely essential to the presently described inventive concept. FIG. 28 by contrast illustrates the arrangement of the present invention when secured to the vertebral bodies and from which it will be appreciated that it can provide a truly “no profile” method of securing an implant which reduces and possibly eliminates the problems of the prior art arrangements.

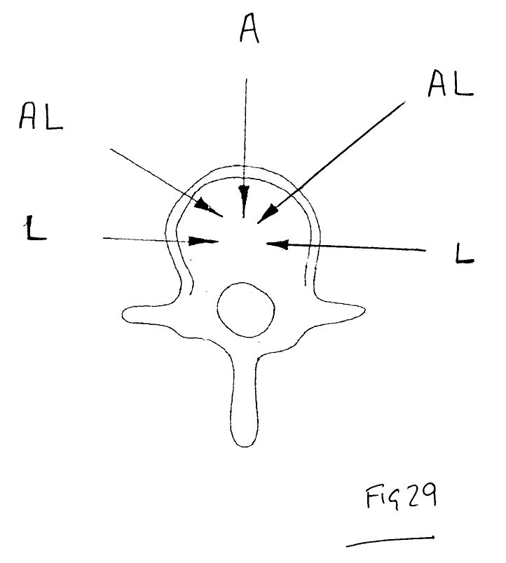

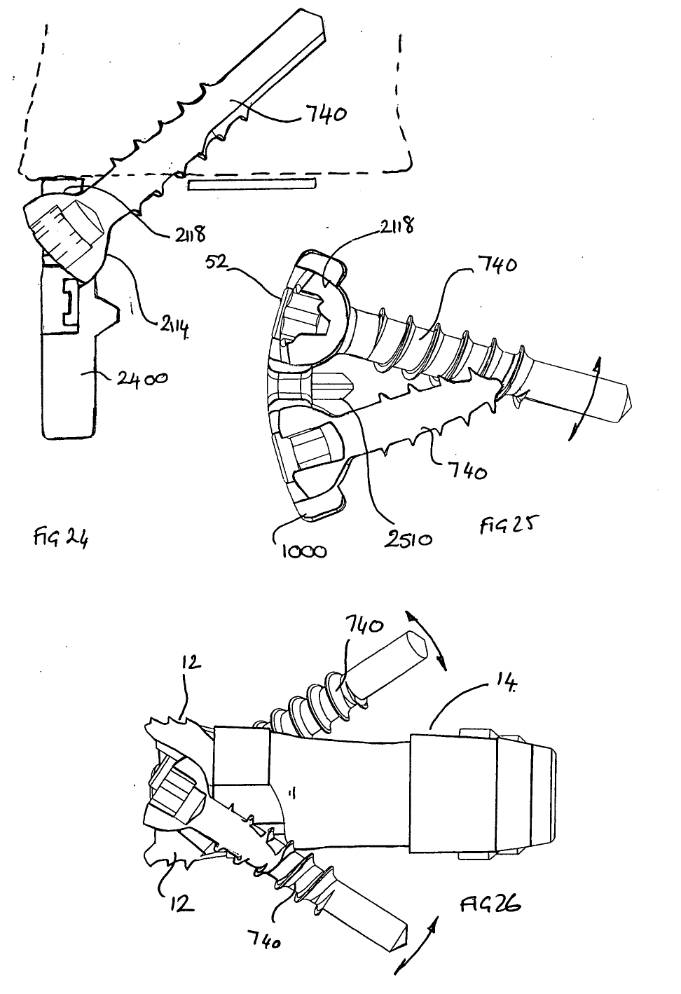

FIG. 29 illustrates the different approaches that may be employed in the placement of an implant where A defines the anterior approach, AL the anterior lateral approach and L the lateral approach.

### EXAMPLE 5

### Implant Materials

Embodiments of the present invention may implement various bioactive and biocompatible implant materials for making the implant components. In exemplary embodiments, the materials used are capable of withstanding large dynamic, compressive loads, encountered in the spine. Moreover, the implant materials used with embodiments of the present invention may implement radiopacity materials known in the art.

In some embodiments, the materials for making components of a implant are comprised of a biocompatible, hardenable polymeric matrix reinforced with bioactive and non-bioactive fillers. The materials can be comprised of about 10% to about 90% by weight of the polymeric matrix and about 10% to about 90% by weight of one or more fillers. The materials can also be comprised of about 20% to about 50% by weight of the polymeric matrix and about 50% to about 80% by weight of one or more fillers. In order to promote bone bonding to the implants, the implants of the present invention can be comprised of a bioactive material that can comprise a polymeric blended resin reinforced with bioactive ceramic fillers. Examples of such bioactive materials can be found, for example, in U.S. Pat. Nos. 5,681,872 and 5,914,356 and pending U.S. application Ser. No. 10/127,947, which is assigned to the assignee of the present invention and incorporated herein by reference in its entirety.

Also discussed herein is the use of bone ingrowth materials which are disposed within the various cavities of the embodiments, and/or used as coating the components. Further, in alternate embodiments, bone ingrowth materials are used for making the actual structural components. Bone ingrowth materials may comprise known bioactive materials including but not limited to BMP or other suitable growth factors, allograft bone with/without stem cell enrichment, calcium phosphate, and/or autograft bone. See U.S. Pat. Nos. 6,899,107 and 6,758,849 for general information on osteoinductive, osteoconductive and/or osteogenic materials and implants.

The disclosures of the cited patent documents, publications and references are incorporated herein in their entirety to the extent not inconsistent with the teachings herein. It should be understood that the examples and embodiments described herein are for illustrative purposes only and that various modifications or changes in light thereof will be suggested to persons skilled in the art and are to be included within the spirit and purview of this application.

What is claimed is:

1. A plate useful for implantation within an intervertebral space, said plate comprising a side perimeter surface, a top surface, and a bottom surface; and at least one channel defined through said plate, wherein said at least one channel is configured to direct an elongate bone fixator in a generally superior or generally inferior direction and wherein said plate is load bearing along a vector between top surface and bottom surface and said plate comprises a geometric dimension mimicking anatomy, lordosis and/or height of said intervertebral space.

2. The implant of claim 1, wherein said at least one channel is configured to allow said elongate bone fixator to freely slide therein so as to allow an interfragmentary association with said elongate bone fixator.

3. The plate of claim 1 further comprising at least one locking component movably affixed thereto and proximate to said at least one said channel such that said locking component can be shifted to block at least a portion of its proximate channel.

4. The plate of claim 1, wherein said at least one channel comprises a first channel defined in said plate according to a vector beginning at said side perimeter surface and directed into a superior vertebral body and a second channel defined in said plate according to a vector beginning at said side perimeter surface and directed into an inferior vertebral body.

5. The plate of claim 1, wherein a cross section of a front-back longitudinal plane of the plate generally tapers from one to the other.

6. The plate of claim 1, wherein a cross section of a lateral-lateral longitudinal plane of the plate generally tapers from one end to another.

7. The plate of claim 1, wherein said at least one channel is configured to allow angular variability of 20 degrees or less on either side of a central axis of said at least one channel for an elongate bone fixator situated in said at least one channel.

8. The plate of claim 1, wherein the plate is arcuate from a lateral end to another.

9. The plate of claim 1, wherein said spacer top surface and bottom surface comprise at least one projection to assist in gripping a superior and inferior vertebral body, respectively.

10. The plate of claim 9, wherein said at least one projection is at least one serration, at least one ridge, at least one fin, or at least one knob, or a combination thereof.

11. An interbody implant comprising
a plate comprising a side perimeter surface top surface and bottom surface; and at least one channel defined through said plate; and
a spacer, said spacer comprising a top surface and bottom surface and side perimeter surface, wherein said side perimeter surface comprises at least one fixator portal; and wherein a portion of said plate side perimeter surface is configured to rest adjacently to at least a portion of said spacer side surface such that said at least one channel is aligned with said at least one fixator portal.

12. The interbody implant of claim 11, wherein said spacer further comprises an interlocking aperture defined therethrough and said plate comprises an interlocking aperture, wherein said spacer interlocking aperture and said plate interlocking aperture are aligned.

13. The interbody implant of claim 12, further comprising an interlocking member passing through said first and second interlocking apertures which interlocks said plate to said spacer.

14. The interbody implant of claim 11, wherein said plate further comprises at least one locking component movably affixed thereto and being proximate to said at least one channel such that said at least one locking component can be shifted to block at least a portion of said at least one channel.

15. The interbody implant of claim 11, wherein said plate top surface and plate bottom surface comprise at least one projection to assist in gripping a superior and inferior vertebral body, respectively.

16. The interbody implant of claim 15, wherein said at least one projection comprises at least one serration, at least one ridge, at least one fin, at least one keel or at least one knob, or a combination thereof.

17. The interbody implant of claim 11, wherein said spacer top surface and bottom surface comprise at least one projection to assist in gripping a superior and inferior vertebral body, respectively.

18. The interbody implant of claim 11, wherein said at least one channel is configured to direct an elongate bone fixator in a generally superior or generally inferior direction.

19. The interbody implant of claim 11, wherein said at least one channel is configured to provide an elongate fixator with an angular variability of adjustment of 25 degrees or less on either side of a central axis of said at least one channel.

20. The interbody implant of claim 19, wherein said anterior body portion is generally arcuate and said posterior body portion is generally straight.

21. The interbody implant of claim 20 wherein at least a portion of said plate side perimeter surface is arcuate and is positioned adjacent to at least a portion of said anterior body portion.

22. An interbody implant according to claim 11 especially useful for a lateral surgical approach wherein said spacer side perimeter surface comprises a first lateral end and a second lateral end, said first lateral end comprising a lateral side surface and into which said at least one fixator portal is defined; and
wherein said plate rests against said first lateral end.

23. The interbody implant of claim 22, wherein said plate further comprises at least one locking component movably affixed thereto and proximate to said at least one channel, such that said at least one locking component can be shifted to block at least a portion of said at least one channel.

24. The interbody implant of claim 22, wherein said first lateral end comprises a first interlocking aperture defined therein; and wherein said plate comprises a second interlocking aperture defined therein; said first and second interlocking apertures being aligned and further comprising an interlocking member passing through said first and second interlocking apertures which interlocks said plate to said spacer.

25. The interbody implant of claim 22, wherein said plate top surface and plate bottom surface comprise at least one projection extending therefrom to assist in gripping a superior and inferior vertebral body surface, respectively.

26. The interbody implant of claim 25, wherein said wherein a cross section of a lateral-lateral longitudinal plane of the plate generally tapers from one end to another.

27. The interbody implant of claim 22, wherein said spacer top surface and bottom surface comprise at least one projection to assist in gripping a superior and inferior vertebral body, respectively.

28. The interbody implant of claim 27, wherein said at least one projection comprises at least one serration, at least one ridge, at least one fin, at least one keel or at least one knob, or a combination thereof.

29. The interbody implant of claim 22, wherein said at least one contoured portion of said first lateral end is generally curved.

30. The interbody implant of claim 29, wherein said spacer component comprises a generally straight posterior side surface.

31. The interbody implant of claim 11 wherein said spacer comprises an anterior to posterior longitudinal plane cross-section that tapers down from a heightened portion to anterior and posterior sides.

32. The interbody implant of claim 12 wherein said unitary implant comprises an anterior to posterior side cross-section that tapers down from anterior to posterior side.

33. The interbody implant of claim 11, wherein said spacer comprises a cavity for disposing bone growth material, said cavity confined with said side perimeter surface.

34. The interbody implant of claim 22, wherein said spacer comprises a cavity for disposing bone growth material, said cavity confined with said side perimeter surface.

35. The interbody implant of claim 11, wherein said plate comprises a first indention defined on said plate top surface and a second indention defined on said plate bottom surface, wherein said indentations assist with holding said plate.

36. A kit for facilitating spinal surgery comprising a plurality of spacer components having differing dimensions, each spacer component comprising a top surface and bottom surface and side perimeter surface having at least one fixator portal said spacer side perimeter surface; and
a plurality of plate components having differing dimensions, each plate component comprising a side perimeter surface, a top surface and a bottom surface, wherein said plate comprises at least one channel configured to allow angular variability of an elongate bone fixator of 25 degrees or less on either side of a central axis of said at least one channel; and wherein at least one of said plurality of plates comprises a side perimeter surface having at least a portion that is configured to rest adjacently against at least a portion of at least one spacer side perimeter surface of at least one of said plurality of spacers such that said at least one channel overlays said at least one fixator portal.

37. The kit of claim 36, wherein said spacer comprises a first interlocking aperture defined therethough and said plate comprises a second interlocking aperture defined therethrough and wherein said first and second interlocking apertures are aligned when said plate and spacer are brought together.

38. A method for surgically implanting an implant in an intervertebral space between a superior and inferior vertebra, said method comprising
positioning into said intervertebral space a spacer comprising a top surface and bottom surface and side perimeter surface, wherein said side perimeter surface comprises at least one fixator portal defined therein;;
positioning into said intervertebral space a plate comprising a side perimeter surface, top surface and bottom surface, wherein said plate comprises at least one channel; wherein said plate has no-profile outside of said intervertebral space and wherein a portion of said plate side perimeter surface is configured to rest adjacently against at least a portion of said spacer side perimeter surface such that said at least one channel overlays said at least one fixator portal;
securing a fastener through said at least one channel/ portal and into said superior or inferior vertebra.

39. The method of claim 38, wherein said spacer comprises a first interlocking aperture defined therethough and said plate comprises a second interlocking aperture defined therethrough and wherein said first and second interlocking apertures are aligned when said plate and spacer are brought together.

40. The method of claim 38, wherein said plate further comprises at least one locking component movably affixed thereto; said at least one locking component proximate to said at least one channel, such that said at least one locking component can be shifted to block at least a portion of said at least one channel.

41. The method of claim 39, further comprising interlocking said spacer to said plate by inserting an interlocking member through said first and second interlocking apertures.

42. A method for surgically implanting an implant into an intervertebral space between a superior and inferior vertebra, said method comprising
positioning into said intervertebral space a plate comprising a side perimeter surface, top surface and bottom surface, wherein said plate comprises at least one channel through said plate wherein said at least one channel is configured to direct an elongate bone fixator in a generally superior or generally inferior direction and wherein said plate is load bearing along a vector between top surface and bottom surface and said plate comprises a geometric dimension mimicking anatomy, lordosis and/or height of said intervertebral space; and
securing a fastener through said at least one channel and into said superior or inferior vertebra.

43. The method of claim 42, wherein said plate is positioned so as to have no profile extending out of said intervertebral space.

44. The method of claim 42, wherein said at least one channel is configured to provide an elongate fixator with an angular variability of adjustment of 25 degrees or less on either side of a central axis of said at least one channel.

45. A bone fixation device comprising:
a screw having a thread portion and a head portion, said head portion having a first surface and a second surface; and
a housing having a first side and a second side and having an aperture therein for receiving said screw and further having a seating portion for receiving the second surface of said head portion wherein:
said aperture comprises a tapered aperture expanding away from said first side of said housing;
said first surface of said head includes a circumferentially extending surface of radius Rc; and
said apparatus includes a locking mechanism on said housing and movable between a first closed position where it engages with said circumferentially extending surface thereby to retain said screw in said housing and a second position where it acts to disengage therefrom to allow said screw to be withdrawn form said aperture.

46. A bone fixation device as claimed in claim 45 wherein said second surface of said head comprises a convex surface having a radius of curvature Ra.

47. A bone fixation device as claimed in claim 45 wherein said seating portion comprises a convex surface having a radius of curvature Rb.

48. A bone fixation device as claimed in claim 47 wherein the radius of curvature of said head portion and said seating portion are substantially the same as each other.

49. A bone fixation device as claimed in claim 45 wherein said locking mechanism comprises a rotatable member rotatable about an axis P between said first and said second positions.

50. A bone fixation device as claimed in claim 45 wherein said locking mechanism comprises a slidable member slidable between said first and said second positions.

51. A bone fixation device as claimed in claim 45 wherein said locking mechanism comprises a friction locking mechanism for frictionally engaging with said circumferentially extending surface.

52. A bone fixation device as claimed in claim 45 wherein said housing comprises a vertebral cage.

53. A bone fixation device as claimed in claim 45 wherein said housing comprises a cage plate for retaining a vertebral cage within a vertebral cavity.
